SEQUENCE LISTING (2 sequences)

SEQ ID NO: 1 - DNA, 1086 bp, Arabidopsis thaliana
atggagtcaagtgatcgttcaagtcaagcaaaagctttcgacgagacaaaaaccggcgtgaaagggcttgtggcttcgggaatcaaagagattccagccatgttccatacacctccggatactctaacaagcctgaaacaaacagcaccaccttcgcagcagctgacgatccccacggtggatctgaaaggaggaagcatggatttgatatcgcggcggagcgtggtggagaagattggagacgctgcggagagatggggattcttccaggtggtgaatcatgggatctcggtggaggtgatggagaggatgaaagaagggattcgcaggtttcacgagcaggacccggaagtgaagaaacggttctactctagggatcacactagagatgtgctttactacagcaacatcgatctccacacttgtaataaggctgcaaattggagagatacgctcgcctgttacatggcccccgatcctcccaagttacaggacttgcccgcggtttgcggggagattatgatggagtactcaaagcaactaatgactttaggtgaatttctctttgagcttctatctgaggctttgggattaaaccctaatcacctcaaggacatgggctgtgccaagtctcatatcatgtttggccaatactatccaccttgccctcagcctgaccttactttaggcataagcaagcacaccgatttctcgtttatcaccattcttcttcaggacaatatcggagggcttcaagttatccatgaccaatgctgggttgatgtttctcctgtccctggcgcccttgtcattaacatcggagatcttctccagcttataagcaatgacaaattcattagcgcggagcatagggtgatagcaaatggatcttctgaaccgcggatttcaatgccatgtttcgtcagcacgttcatgaagccgaatccacgaatatatggacccatcaaagaacttttgtcagaacaaaaccctgccaagtatagagacttaaccatcaccgagttttcaaacaccttcaggtcccaaacgatcagtcaccctgcgttacaccatttcaggatctga

SEQ ID NO: 2 - protein, 361 aa, Arabidopsis thaliana
MESSDRSSQAKAFDETKTGVKGLVASGIKEIPAMFHTPPDTLTSLKQTAPPSQQLTIPTVDLKGGSMDLISRRSVVEKIGDAAERWGFFQVVNHGISVEVMERMKEGIRRFHEQDPEVKKRFYSRDHTRDVLYYSNIDLHTCNKAANWRDTLACYMAPDPPKLQDLPAVCGEIMMEYSKQLMTLGEFLFELLSEALGLNPNHLKDMGCAKSHIMFGQYYPPCPQPDLTLGISKHTDFSFITILLQDNIGGLQVIHDQCWVDVSPVPGALVINIGDLLQLISNDKFISAEHRVIANGSSEPRISMPCFVSTFMKPNPRIYGPIKELLSEQNPAKYRDLTITEFSNTFRSQTISHPALHHFRI

(19) United States
(12) Patent Application Publication
(10) Pub. No.: US 2003/0186278 A1
(43) Pub. Date: Oct. 2, 2003

(54) Methods for the identification of inhibitors of 1-aminocyclopropane-1-carboxylate oxidase expression or activity in plants

(76) Inventors: Joseph Mitchell, Raleigh, NC (US); Adel Zayed, Durham, NC (US); Robert Ascenzi, Cary, NC (US); Douglas Boyes, Chapel Hill, NC (US); Rao Mulpuri, Apex, NC (US); Neil Hoffman, Chapel Hill, NC (US); Susanne Kjemtrup, Chapel Hill, NC (US); Keith Davis, Durham, NC (US); Carol Hamilton, Apex, NC (US); Jeffrey Woessner, Hillsborough, NC (US); Jorn Gorlach, Manchester, NJ (US)

Correspondence Address: PARADIGM GENETICS, INC, 108 ALEXANDER DRIVE, P O BOX 14528, RTP, NC, 27709-4528, US

(21) Appl. No.: 10/316,306

(22) Filed: Dec. 11, 2002

Related U.S. Application Data
(60) Provisional application No. 60/339,250, filed on Dec. 11, 2001.

(51) Int. Cl. C12Q 1/68; C12Q 1/26; A01N 25/00

(52) U.S. Cl. 435/6; 435/25; 504/116.1

(57) ABSTRACT

The present inventors have discovered that 1-Aminocyclopropane-1-Carboxylate Oxidase (ACC) is essential for plant growth. Specifically, the inhibition of ACC gene expression in plant seedlings results in reduced and severely stunted growth, and chlorosis. Thus, ACC can be used as a target for the identification of herbicides. Accordingly, the present invention provides methods for the identification of compounds that inhibit ACC expression or activity, comprising: contacting a compound with an ACC and detecting the presence and/or absence of binding between said compound and said an ACC, or detecting a decrease in ACC expression or activity. The methods of the invention are useful for the identification of herbicides.

This application claims the benefit of U.S. Provisional Application No. 60/339,250, filed Dec. 11, 2001, the content of which is hereby incorporated in its entirety.

### FIELD OF THE INVENTION

The invention relates generally to plant molecular biology. In particular, the invention relates to methods for the identification of herbicides.

### BACKGROUND OF THE INVENTION

Ethylene has profound effects on many developmental events and environmental responses of plants (Yang and Hoffman (1984) Annu Rev Plant Physiol 35: 155-189). Endogenous production of ethylene increases during certain stages of growth and development, such as seed germination, fruit ripening, and leaf and flower senescence and abscission, and in response to drought, flooding, physical wounding, chilling injury, pathogen infection, and chemical inducers (Yang and Hoffman (1984) Annu Rev Plant Physiol 35: 155-189; Theologis (1992) Cell 70: 181-4 (PMID: 1638627)). In higher plants, ethylene is biosynthesized from methionine by a well-defined pathway in which 1-Aminocyclopropane-1-Carboxylic Acid Synthase and 1-Aminocyclopropane-1-Carboxylic Acid Oxidase catalyze the reactions from S-adenosylmethionine to 1-aminocyclopropane-1-carboxylic acid and 1-aminocyclopropane-1-carboxylic acid to ethylene, respectively (Bleecker and Kende (2000) Annu Rev Cell Dev Biol 16: 1-18 (PMID: 11031228)). With advancement in molecular biology techniques, cDNA and genomic clones for both enzymes have been isolated from various plant species, and both enzymes appear to be encoded by multigene families.

Using these cDNA clones, expression of individual members has been characterized in different tissues and in response to specific stimuli known to induce ethylene biosynthesis (Kende (1993) Annu Rev Plant Physiol Plant Mol Biol 44: 283-307; Zarembinski and Theologis (1994) Plant Mol Biol 26:1579-97 (PMID: 7858205); Fluhr and Mattoo (1996) CRC Crit Rev Plant Sci 15: 479-523). Fruits have been classified as climacteric and non-climacteric on the basis of their patterns of respiration and ethylene production during maturation and ripening (Biale and Young (1981) Respiration and ripening in fruits: retrospect and prospect. In J Friend, MJC Rhodes, eds, Recent Advances in the Biochemistry of Fruits and Vegetables. AcademicPress, London, pp 1-39). In climacteric fruits, it has been accepted that ethylene plays an important role in ripening in that a massive production of ethylene commences at the onset of the respiratory climacteric period, and exogenously applied ethylene induces ripening and endogenous ethylene production. In ripening climacteric fruits, both 1-Aminocyclopropane-1-Carboxylic Acid Synthase and 1-Aminocyclopropane-1-Carboxylic Acid Oxidase are induced and contribute to the regulation of ethylene biosynthesis (Yang and Hoffman (1984) Annu Rev Plant Physiol 35: 155-189).

Expression of 1-Aminocyclopropane-1-Carboxylic Acid Oxidase genes has been investigated in fruits such as tomato (Barry et al. (1996) Plant J 9: 525-35 (PMID: 8624515); Nakatsuka et al. (1998) Plant Physiol 118: 1295-305 (PMID: 9847103)), apple (Ross et al. (1992) Plant Mol Biol 19: 231-8 (PMID: 1377961)), melon (Balague et al. (1993) Eur J Biochem 212: 27-34 (PMID: 8444161); Yamamoto et al. (1995) Plant Cell Physiol 36: 591-596; Lasserre et al. (1996) Mol Gen Genet 251: 81-90 (PMID: 8628251)), kiwi (Whittaker et al. (1997) Plant Mol Biol 34: 45-55 (PMID: 9177311)), pear (Lelievre et al. (1997b) Postharvest Biol Technol 5: 11-17), cucumber (Shiomi et al. (1998) Jpn Soc Hortic Sci 67: 685-692), passion fruit (Mita et al. (1998) Plant Cell Physiol 39: 1209-17 (PMID: 9891418)), and banana (Huang et al. (1997) Biochem Mol Biol Int 41: 941-50 (PMID: 9137825); Lopez-Gomez et al. (1997) Plant Sci 123: 123-131).

To date there do not appear to be any publications describing lethal effects of over-expression, antisense expression or knock-out of this gene in plants. Thus, the prior art has not suggested that ACC is essential for plant growth and development. It would be desirable to determine the utility of this enzyme for evaluating plant growth regulators, especially herbicide compounds, to include, but not limited to, determinations in climacteric and/or non-climacteric fruit-producing plants.

### SUMMARY OF THE INVENTION

Surprisingly, the present inventors have discovered that antisense expression of an ACC cDNA in Arabidopsis causes developmental abnormalities, reduced and severely stunted growth, and chlorosis. Thus, the present inventors have discovered that ACC is essential for normal seed development and growth, and can be used as a target for the identification of herbicides. Accordingly, the present invention provides methods for the identification of compounds that inhibit ACC expression or activity, comprising: contacting a candidate compound with an ACC and detecting the presence or absence of binding between said compound and said ACC, or detecting a decrease in ACC expression or activity. The methods of the invention are useful for the identification of herbicides.

### BRIEF DESCRIPTION OF THE DRAWINGS

FIG. 1 shows the 1-Aminocyclopropane-1-Carboxylate Oxidase reaction.

### DETAILED DESCRIPTION OF THE INVENTION

Definitions

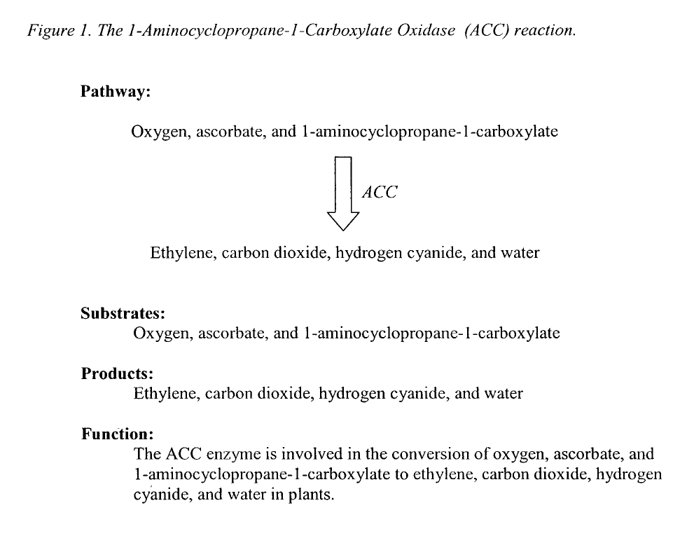

As used herein, the term “1-Aminocyclopropane-1-Carboxylate Oxidase (EC 1.4.3.-)” is synonymous with “Ethylene Forming Enzyme” and “ACC” and refers to an enzyme that catalyses the conversion of oxygen, ascorbate, and 1-aminocyclopropane-1-carboxylate to ethylene, carbon dioxide, hydrogen cyanide, and water, as shown in FIG. 1, and as included herein as the protein of SEQ ID NO: 2 and/or its encoding cDNA, SEQ ID NO: 1.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

As used herein, the term “cDNA” means complementary deoxyribonucleic acid.

As used herein, the term “DNA” means deoxyribonucleic acid.

As used herein, the term “dI” means deionized.

As used herein, the term “ELISA” means enzyme-linked immunosorbent assay.

As used herein, the term “GUS” means β-glucouronidase.

The term “herbicide”, as used herein, refers to a compound that may be used to kill or suppress the growth of at least one plant, plant cell, plant tissue or seed.

As used herein, the term “HPLC” means high pressure liquid chromatography.

The term “inhibitor”, as used herein, refers to a chemical substance that inactivates the enzymatic activity of ACC. The inhibitor may function by interacting directly with the enzyme, a cofactor of the enzyme, the substrate of the enzyme, or any combination thereof.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

As used herein, the term “LB” means Luria-Bertani media.

As used herein, the term “mRNA” means messenger ribonucleic acid.

As used herein, the term “Ni” refers to nickel.

As used herein, the term “Ni-NTA” refers to nickel sepharose.

As used herein, the term “PCR” means polymerase chain reaction.

The “percent (%) sequence identity” between two polynucleotide or two polypeptide sequences can be determined according to the either the BLAST program (Basic Local Alignment Search Tool, Altschul and Gish (1996) Meth Enzymol 266: 460-480; Altschul (1990) J Mol Biol 215: 403-410) in the Wisconsin Genetics Software Package (Devererreux et al. (1984) Nucl Acid Res 12: 387), Genetics Computer Group (GCG), Madison, Wis. (NCBI, Version 2.0.11, default settings) or using Smith Waterman Alignment (Smith and Waterman (1981) Adv Appl Math 2:482) as incorporated into GeneMatcher Plus™ (Paracel, Inc., using the default settings and the version current at the time of filing). It is understood that for the purposes of determining sequence identity when comparing a DNA sequence to an RNA sequence, a thymine nucleotide is equivalent to an uracil nucleotide.

As used herein, the term “PGI” means plant growth inhibition.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

As used herein, the term “RNA” means ribonucleic acid.

As used herein, the term “SDS” means sodium dodecyl sulfate.

As used herein, the term “SDS-PAGE” means sodium dodecyl sulfate-polyacrylimide gel electrophoresis.

The term “specific binding” refers to an interaction between ACC and a molecule or compound, wherein the interaction is dependent upon the primary amino acid sequence or the conformation of ACC.

As used herein, the term “TATA box” refers to a sequence of nucleotides that serves as the main recognition site for the attachment of RNA polymerase in the promoter region of eukaryotic genes. Located at around 25 nucleotides before the start of transcription, it consists of the seven-base consensus sequence TATAAAA, and is analogous to the Pribnow box in prokaryotic promoters.

As used herein, the term “TLC” means thin layer chromatography.

Embodiments of the Invention

The present inventors have discovered that inhibition of ACC gene expression strongly inhibits the growth and development of plant seedlings. Thus, the inventors are the first to demonstrate that ACC is a target for herbicides.

Accordingly, the invention provides methods for identifying compounds that inhibit ACC gene expression or activity. Such methods include ligand binding assays, assays for enzyme activity and assays for ACC gene expression. Any compound that is a ligand for ACC, other than its substrates, oxygen, ascorbate, and 1-aminocyclopropane-1-carboxylate, may have herbicidal activity. For the purposes of the invention, “ligand” refers to a molecule that will bind to a site on a polypeptide. The compounds identified by the methods of the invention are useful as herbicides.

Thus, in one embodiment, the invention provides a method for identifying a compound as a candidate for a herbicide, comprising:

a) contacting an ACC with said compound; and

b) detecting the presence and/or absence of binding between said compound and said ACC,

wherein binding indicates that said compound is a candidate for a herbicide.

By “ACC” is meant any enzyme that catalyzes the interconversion of oxygen, ascorbate, and 1-aminocyclopropane-1-carboxylate with ethylene, carbon dioxide, hydrogen cyanide, and water. The ACC may have the amino acid sequence of a naturally occurring ACC found in a plant, animal or microorganism, or may have an amino acid sequence derived from a naturally occurring sequence. Preferably the ACC is a plant ACC. The cDNA (SEQ ID NO: 1) encoding the ACC protein or polypeptide (SEQ ID NO: 2) can be found herein as well as in the TIGR database at locus At1g03410.

By “plant ACC” is meant an enzyme that can be found in at least one plant, and which catalyzes the interconversion of oxygen, ascorbate, and 1-aminocyclopropane-1-carboxylate with ethylene, carbon dioxide, hydrogen cyanide, and water. The ACC may be from any plant, including monocots and dicots.

In one embodiment, the ACC is an Arabidopsis ACC. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis ACC is from Arabidopsis thaliana.

In various embodiments, the ACC can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of an ACC polypeptide may be used in the methods of the invention. The fragments comprise at least 10 consecutive amino acids of an ACC. Preferably, the fragment comprises at least 15, 20, 25, 30, 35, 40, 50, 60, 70, 80, 90 or at least 100 consecutive amino acids residues of an ACC. In one embodiment, the fragment is from an Arabidopsis ACC. Preferably, the fragment contains an amino acid sequence conserved among plant 1-Aminocyclopropane-1-Carboxylate Oxidases. Such conserved fragments are identified in Grima-Pettenuti et al. (1993) Plant Mol Biol 21: 1085-1095 and Taveres et al. (2000), supra. Those skilled in the art could identify additional conserved fragments using sequence comparison software.

Polypeptides having at least 80% sequence identity with a plant ACC are also useful in the methods of the invention. Preferably, the sequence identity is at least 85%, more preferably the identity is at least 90%, most preferably the sequence identity is at least 95% or 99%.

In addition, it is preferred that the polypeptide has at least 50% of the activity of a plant ACC. More preferably, the polypeptide has at least 60%, at least 70%, at least 80% or at least 90% of the activity of a plant ACC. Most preferably, the polypeptide has at least 50%, at least 60%, at least 70%, at least 80%, or at least 90% of the activity of the A. thaliana ACC protein.

Thus, in another embodiment, the invention provides a method for identifying a compound as a candidate for a herbicide, comprising:

a) contacting said compound with at least one polypeptide selected from the group consisting of: a plant ACC, a polypeptide comprising at least ten consecutive amino acids of a plant ACC, a polypeptide having at least 85% sequence identity with a plant ACC, and a polypeptide having at least 80% sequence identity with a plant ACC and at least 50% of the activity thereof; and

b) detecting the presence and/or absence of binding between said compound and said polypeptide,

wherein binding indicates that said compound is a candidate for a herbicide.

Any technique for detecting the binding of a ligand to its target may be used in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with an ACC protein or a fragment or variant thereof, the unbound protein is removed and the bound ACC is detected. In a preferred embodiment, bound ACC is detected using a labeled binding partner, such as a labeled antibody. In a variation of this assay, ACC is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for a herbicide, it can be tested for the ability to inhibit ACC enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with said herbicide candidate and detecting the presence or absence of a decrease in the growth or viability of said plant or plant cells.

A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be at decreased by at least 40%. More preferably, the growth or viability will be decreased by at least 50%, 75%, or at least 90% or more. Methods for measuring plant growth and cell viability are known to those skilled in the art. It is possible that a candidate compound may have herbicidal activity only for certain plants or certain plant species.

The ability of a compound to inhibit ACC activity can be detected using in vitro enzymatic assays in which the disappearance of a substrate or the appearance of a product is directly or indirectly detected. ACC catalyzes the irreversible or reversible reaction of oxygen, ascorbate, and 1-aminocyclopropane-1-carboxylate to ethylene, carbon dioxide, hydrogen cyanide, and water. Methods for detection of oxygen, ascorbate, and 1-aminocyclopropane-1-carboxylate, and/or ethylene, carbon dioxide, hydrogen cyanide, and water, include spectrophotometry, mass spectroscopy, thin layer chromatography (TLC) and reverse phase HPLC.

Thus, the invention provides a method for identifying a compound as a candidate for a herbicide, comprising:

a) contacting an oxygen, ascorbate, and 1-aminocyclopropane-1-carboxylate with ACC;

b) contacting said oxygen, ascorbate, and 1-aminocyclopropane-1-carboxylate with ACC and said candidate compound; and

c) determining the concentration of ethylene, carbon dioxide, hydrogen cyanide, and/or water after the contacting of steps (a) and (b).

If a candidate compound inhibits ACC activity, a higher concentration of the substrates (Oxygen, ascorbate, and 1-aminocyclopropane-1-carboxylate) and a lower level of the products (Ethylene, carbon dioxide, hydrogen cyanide, and water) will be detected in the presence of the candidate compound (step b) than that detected in the absence of the compound (step a).

Preferably the ACC is a plant ACC. Enzymatically active fragments of a plant ACC are also useful in the methods of the invention. For example, a polypeptide comprising at least 100 consecutive amino acid residues of a plant ACC may be used in the methods of the invention. In addition, a polypeptide having at least 80%, 85%, 90%, 95%, 98% or at least 99% sequence identity with a plant ACC may be used in the methods of the invention. Preferably, the polypeptide has at least 80% sequence identity with a plant ACC and at least 50%, 75%, 90% or at least 95% of the activity thereof.

Thus, the invention provides a method for identifying a compound as a candidate for a herbicide, comprising:

a) contacting oxygen, ascorbate, and 1-aminocyclopropane-1-carboxylate with a polypeptide selected from the group consisting of: a polypeptide having at least 85% sequence identity with a plant ACC, a polypeptide having at least 80% sequence identity with a plant ACC and at least 50% of the activity thereof, and a polypeptide comprising at least 100 consecutive amino acids of a plant ACC;

b) contacting said oxygen, ascorbate, and 1-aminocyclopropane-1-carboxylate with said polypeptide and said compound; and

c) determining the concentration of ethylene, carbon dioxide, hydrogen cyanide, and/or water after the contacting of steps (a) and (b).

Again, if a candidate compound inhibits ACC activity, a higher concentration of the substrates (Oxygen, ascorbate, and 1-aminocyclopropane-1-carboxylate) and a lower level of the products (Ethylene, carbon dioxide, hydrogen cyanide, and water) will be detected in the presence of the candidate compound (step b) than that detected in the absence of the compound (step a).

For the in vitro enzymatic assays, ACC protein and derivatives thereof may be purified from a plant or may be recombinantly produced in and purified from a plant, bacteria, or eukaryotic cell culture. Preferably ACC proteins are produced using a baculovirus or E. coli expression system. Methods for purifying ACC may be found in Thrower et al. (2001) Biochemistry 40: 9717-24 (PMID: 11583172). Other methods for the purification of ACC proteins and polypeptides are known to those skilled in the art.

As an alternative to in vitro assays, the invention also provides plant and plant cell based assays. In one embodiment, the invention provides a method for identifying a compound as a candidate for a herbicide, comprising:

a) measuring the expression of ACC in a plant or plant cell in the absence of said compound;

b) contacting a plant or plant cell with said compound and measuring the expression of ACC in said plant or plant cell; and

c) comparing the expression of ACC in steps (a) and (b).

A reduction in ACC expression indicates that the compound is a herbicide candidate. In one embodiment, the plant or plant cell is an Arabidopsis thaliana plant or plant cell.

Expression of ACC can be measured by detecting the ACC primary transcript or mRNA, ACC polypeptide or ACC enzymatic activity. Methods for detecting the expression of RNA and proteins are known to those skilled in the art. (See, for example, Current Protocols in Molecular Biology, Ausubel et al., eds., Greene Publishing and Wiley-Interscience, New York, 1995). However, the method of detection is not critical to the invention. Methods for detecting ACC RNA include, but are not limited to, amplification assays such as quantitative PCR, and/or hybridization assays such as Northern analysis, dot blots, slot blots, in-situ hybridization, transcriptional fusions using an ACC promoter fused to a reporter gene, bDNA assays, and microarray assays.

Methods for detecting protein expression include, but are not limited to, immunodetection methods such as Western blots, His Tag and ELISA assays, polyacrylamide gel electrophoresis, mass spectroscopy, and enzymatic assays. Also, any reporter gene system may be used to detect ACC protein expression. For detection using gene reporter systems, a polynucleotide encoding a reporter protein is fused in frame with ACC, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2: 1104; Prost et al. (1986) Gene 45: 107-111), β-galactosidase (Nolan et al. (1988) Proc Natl Acad Sci USA 85: 2603-2607), alkaline phosphatase (Berger et al. (1988) Gene 66: 10), luciferase (De Wet et al. (1987) Mol Cell Biol 7: 725-737), β-glucuronidase (GUS), fluorescent proteins, chromogenic proteins and the like. Methods for detecting ACC activity are described above.

Chemicals, compounds, or compositions identified by the above methods as modulators of ACC expression or activity can be used to control plant growth. For example, compounds that inhibit plant growth can be applied to a plant or expressed in a plant to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### Experimental

Plant Growth Conditions

Unless, otherwise indicated, all plants are grown in Scotts Metro-Mix™ soil (the Scotts Company) or a similar soil mixture in an environmental growth room at 22° C., 65% humidity, 65% humidity and a light intensity of ˜100 μ-Em⁻²s⁻¹ supplied over 16 hour day period.

Seed Sterilization

All seeds are surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds are sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ inch down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-Em⁻²s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant Expressing the Driver

The “Driver” is an artificial transcription factor comprising a chimera of the DNA-binding domain of the yeast GAL4 protein (amino acid residues 1-147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of Antisense Expression Cassettes in a Binary Vector

A fragment or variant of an Arabidopsis thaliana cDNA corresponding to SEQ ID NO: 1 was ligated into the PacI/AscI sites of an E. coli/Agrobacterium binary vector in the antisense orientation. This placed transcription of the antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E. coli. Kanamycin resistant clones were selected and purified. DNA was isolated from each clone and characterized by PCR and sequence analysis. The DNA was inserted in a vector that expresses the A. thaliana antisense RNA, which is complementary to a portion of the DNA of SEQ ID NO: 1. This antisense RNA is complementary to the cDNA sequence found in the TIGR database at locus At1g03410. The coding sequence for this locus is shown as SEQ ID NO: 1. The protein encoded by these mRNAs is shown as SEQ ID NO: 2.

The antisense expression cassette and a constitutive chemical resistance expression cassette are located between right and left T-DNA borders. Thus, the antisense expression cassettes can be transferred into a recipient plant cell by agroinfection.

### EXAMPLE 3

### Transformation of Agrobacterium with the Antisense Expression Cassette

The vector was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### EXAMPLE 4

### Construction of an Arabidopsis Antisense Target Plants

The antisense expression cassette was introduced into Arabidopsis thaliana wild-type plants by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial.

One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represented stably transformed T1 plants.

### EXAMPLE 5

### Effect of Antisense Expression in Arabidopsis Seedlings

The T1 antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. The antisense expression of this gene resulted in significantly impaired growth, indicating that this gene represents an essential gene for normal plant growth and development. The transgenic line containing the antisense construct for 1-Aminocyclopropane-1-Carboxylate Oxidase exhibited significant seedling abnormalities. Seedlings showed reduced and severely stunted growth, and chlorosis.

### EXAMPLE 6

### Cloning and Expression Strategies, Extraction and Purfication of the ACC Protein

The following protocol may be employed to obtain the purified ACC protein.

Cloning and Expression Strategies:

An ACC gene can be cloned into E. coli (pET vectors-Novagen), Baculovirus (Pharmingen) and Yeast (Invitrogen) expression vectors containing His/fusion protein tags, and the expression of recombinant protein can be evaluated by SDS-PAGE and Western blot analysis.

Extraction:

Extract recombinant protein from 250 ml cell pellet in 3 mL of extraction buffer by sonicating 6 times, with 6 sec pulses at 4° C. Centrifuge extract at 15000×g for 10 min and collect supernatant. Assess biological activity of the recombinant protein by activity assay.

Purification:

Purify recombinant protein by Ni-NTA affinity chromatography (Qiagen).

Purification protocol: perform all steps at 4° C.:

Use 3 ml Ni-beads (Qiagen)

Equilibrate column with the buffer

Load protein extract

Wash with the equilibration buffer

Elute bound protein with 0.5 M imidazole

### EXAMPLE 7

### Assays for Testing Inhibitors or Candidates for Inhibition of ACC Activity

The enzymatic activity of ACC may be determined in the presence and absence of candidate inhibitors in a suitable reaction mixture, such as described by any of the following known assay protocols:

A. In vivo ACC Oxidase Assay:

Enzyme activity is estimated for in vivo reactions often by ethylene evolution as measured by gas chromatography. Several methods have been described recently, such as those described in Liu et al. (1999) Plant Physiol 121: 1257-66 (PMID: 10594112), and Evensen et al. (1993) Ann Bot 71: 559-566.

B. In vitro ACC Oxidase Assay:

The usual method of estimating enzyme activity is to make an extract of the tissue, partially purify the enzyme, and then measure its activity by supplying the extract with substrate (oxygen, ascorbate, and 1-aminocyclopropane-1-carboxylate) and measuring a product of the reaction (e.g. ethylene). Moya-Leon and John ((1994) J Hortic Sci 69: 243-250), Thrower et al. (2001) Biochemistry 40: 9717-24 (PMID: 11583172), Dong et al. (1992) Proc Natl Acad Sci USA 89: 9789-93 (PMID: 1409700), and Brunhuber et al. (2000) Biochemistry 39: 10730-8 (PMID: 10978157) describe in vitro assays of ACC oxidase.

While the foregoing describes certain embodiments of the invention, it will be understood by those skilled in the art that variations and modifications may be made and still fall within the scope of the invention.

What is claimed is:

1. A method for identifying a compound as a candidate for a herbicide, comprising:
a) contacting a 1-aminocyclopropane-1-carboxylate oxidase (ACC) with a compound; and
b) detecting the presence and/or absence of binding between said compound and said ACC, wherein binding indicates that said compound is a candidate for a herbicide.

2. The method of claim 1, wherein said ACC is a plant ACC.

3. The method of claim 2, wherein said ACC is an Arabidopsis ACC.

4. The method of claim 3, wherein said ACC is SEQ ID. NO: 2.

5. A method for determining whether a compound identified as a herbicide candidate by the method of claim 1 has herbicidal activity, comprising: contacting a plant or plant cells with said herbicide candidate and detecting a change in growth or viability of said plant or plant cells.

6. A method for identifying a compound as a candidate for a herbicide, comprising:
a) selecting a compound that binds to a polypeptide selected from the group consisting of:
i) the polypeptide set forth in SEQ ID NO:2; and
ii) a polypeptide having at least 80% sequence identity with the polypeptide set forth in SEQ ID NO:2; and
b) contacting a plant with said compound to confirm herbicidal activity.

7. A method for determining whether a compound identified as a herbicide candidate by the method of claim 6 has herbicidal activity, comprising: contacting a plant or plant cells with said herbicide candidate and detecting a change in growth or viability of said plant or plant cells.

8. A method for identifying a test compound as a candidate for a herbicide, comprising:
a) contacting oxygen, ascorbate, and 1-aminocyclopropane-1-carboxylate with 1-aminocyclopropane-1-carboxylate oxidase (ACC);
b) contacting said oxygen, ascorbate, and 1-aminocyclopropane-1-carboxylate with ACC and a test compound; and
c) determining the concentration of at least one of oxygen, ascorbate, 1-aminocyclopropane-1-carboxylate, ethylene, carbon dioxide, hydrogen cyanide, and/or water after the contacting of steps (a) and (b), wherein a higher concentration of a substrate (oxygen, ascorbate, and 1-aminocyclopropane-1-carboxylate) and/or a lower level of a product (ethylene, carbon dioxide, hydrogen cyanide, and water) detected in the presence of the candidate compound (step b) than that detected in the absence of the compound (step a) indicates that said compound is a candidate for a herbicide.

9. The method of claim 8, wherein said ACC is a plant ACC.

10. The method of claim 9, wherein said ACC is an Arabidopsis ACC.

11. The method of claim 10, wherein said ACC is SEQ ID. NO: 2.

12. A method for identifying a compound as a candidate for a herbicide, comprising:
a) contacting oxygen, ascorbate, and 1-aminocyclopropane-1-carboxylate with a polypeptide selected from the group consisting of:
i) the polypeptide set forth in SEQ ID NO:2; and
ii) a polypeptide having at least 80% sequence identity with the polypeptide set forth in SEQ ID NO:2; and
b) contacting said oxygen, ascorbate, and 1-aminocyclopropane-1-carboxylate with said polypeptide and said compound; and
c) determining the concentration of at least one of oxygen, ascorbate, 1-aminocyclopropane-1-carboxylate, ethylene, carbon dioxide, hydrogen cyanide, and/or water after the contacting of steps (a) and (b) wherein a higher concentration of a substrate (oxygen, ascorbate, and 1-aminocyclopropane-1-carboxylate) and/or a lower level of a product (ethylene, carbon dioxide, hydrogen cyanide, and water) detected in the presence of the candidate compound (step b) than that detected in the absence of the compound (step a) indicates that said compound is a candidate for a herbicide.

13. A method for identifying a compound as a candidate for a herbicide, comprising:
a) measuring the expression of a 1-aminocyclopropane-1-carboxylate oxidase (ACC) in a plant or plant cell in the absence of said compound;
b) contacting a plant or plant cell with said compound and measuring the expression of said ACC in said plant or plant cell; and
c) comparing the expression of ACC in steps (a) and (b), wherein a lower level of ACC expression indicates that said compound is a candidate for a herbicide.

14. The method of claim 13 wherein said plant or plant cell is an Arabidopsis plant or plant cell.

15. The method of claim 14, wherein said ACC is SEQ ID NO: 2.

16. The method of claim 13, wherein the expression of 1-aminocyclopropane-1-carboxylate oxidase (ACC) is measured by detecting ACC mRNA.

17. The method of claim 13, wherein the expression of 1-aminocyclopropane-1-carboxylate oxidase (ACC) is measured by detecting ACC polypeptide.
